(19) United States
(12) Patent Application Publication
(10) Pub. No.: US 2010/0255989 A1
(43) Pub. Date: Oct. 7, 2010

(54) Process for removing ethene from biological sources using iodine doped titaniumdioxide

(76) Inventors: Raffaella Sartorio, Sarzana (IT); Mara Destro, Bologna (IT)

Correspondence Address: BASF Corporation;Patent Department, 500 White Plains Road, P.O. Box 2005, Tarrytown, NY, 10591, US

(21) Appl. No.: 12/308,354

(22) PCT Filed: Jun. 11, 2007
(86) PCT No.: PCT/EP2007/055688

§ 371 Date: Jun. 9, 2010

(30) Foreign Application Priority Data
Jun. 20, 2006 (EP) 06115703.8

(51) Int. Cl. B65D 85/50; A61L 9/00; A23L 3/28

(52) U.S. Cl. 504/114; 422/4; 426/118; 426/248

(57) ABSTRACT

The instant invention relates to a process for removing ethene from biological sources using iodine doped titaniumdioxide. Further aspects of the invention are polymer compositions containing this iodine doped titanium dioxide, its use as efficient ethene removing additive and the iodine doped titaniumdioxide itself.

The instant invention relates to a process for removing ethene from biological sources using iodine doped titaniumdioxide. Further aspects of the invention are polymer compositions containing this iodine doped titanium dioxide, its use as efficient ethene removing additive and the iodine doped titaniumdioxide itself.

Eliminating ethene gas, which is generated during storage of biological products, such as fruits, flowers and the like, is an effective way to prolong the post-harvest life of fresh vegetables, fruits and cut flowers. The high concentration of ethene gas accelerates the aging of fresh products.

A number of solutions already exist on the market, based on different technologies. For example ethene can be removed by chemical reaction and this is what happens with potassium permanganate based systems. It can also be removed by adsorption, which is the principle function of zeolites, oya stones and other inorganic additives, most often incorporated into plastic packaging films. This is for example described in EP 1 134 022. A further possibility is by means of catalytic filters which purify the air. All these solutions have drawbacks, such as the low activity of inorganic additives in plastic films or the toxicity of permanganate based sachets, the disposal of which is not easy to handle.

The use of filters based on TiO₂ is also quite common to remove ethene and it is applied both during transportation and during fresh produce storage in the warehouse. TiO₂ is a photo-oxidizing catalyst that degrades ethene but it requires a dedicated UV lamp. UV light represents only a small portion of the solar spectrum that reaches the earth. Accordingly the problem to be solved by the present invention is to develop a novel doped TiO₂ having a broader absorption spectrum, therefore, working more efficiently in oxidizing ethene in the presence of ambient visible light.

Titanium dioxide is widely used for the photodecomposition of organic pollutants for several reasons including its low cost, non-toxicity, high stability and high efficiency. However, it has a great limitation due to the high-energy band gap (ca. 3.2 eV for Anatas) and only UV light (below 400 nm, only 4% of the solar spectrum) is able to promote the oxidation process. Thus by properly shifting the onset of its spectrum from the UV light to the visible region it is possible to improve its performance and to exploit a broader part of the solar radiation for the photo-oxidation process. The resulting catalyst is able to work with ambient light.

Doping titanium dioxide allows the lowering of the band gap thus shifting the absorbance (and the excitation process) towards the visible light.

In the past, a way of doping titanium dioxide was by metal ion implantation with transition metals (V, Ni, Cr, Mn or Fe). However, the use of transition metals as dopants has some drawbacks: low thermal stability, expensive procedures and in some cases increase of the recombination process. More recently nitrogen has been reported as effective dopant (“Visible light photocatalysis in nitrogen-doped titanium oxides” Science, 2001, 293, 269-271).

Furthermore Fedorov et all. reported the photodegradation of ethene with visible light using nitrogen doped titanium nanoparticle slurries (Applied Catalysis B: Environmental, 2005, 57, 93-117).

Another method for preparing nitrogen doped titanium dioxide materials, which is very active in the photo-oxidation of organic materials, has been reported by Lee et al. (Chem. Lett., 2005, 34, 660-661).

The instant invention provides an even more efficient material, namely a highly active visible light responsive titanium dioxide which is doped with iodine. The material is obtained by hydrolysis of a titanium alkoxyde in the presence of iodic acid.

One aspect of the invention is a process for removing ethene from a gas atmosphere, comprising

bringing into contact a iodine doped titaniumdioxide

with a gas atmosphere containing at least partly ethene and irradiating the iodine doped titaniumdioxide with light of a wavelength from 300 to 700 nm.

Preferably the iodine doped titaniumdioxide is exposed to light of a wavelength between 300 and 500 nm.

The main crystal modifications of TiO₂ are Rutil and Anatas. In principal the Anatas modification is preferred for the iodine doped titaniumdioxid, however in most cases there exists a mixture of the Rutil and Anatas modification.

As plants are still alive after being harvested, various physiological effects such as respiration effect, transpiration effect, mold growth and putrefaction under the action of microorganisms, etc. may take place and accelerate the loss of freshness of the plants. In addition, plants evolve ethene, a kind of plant hormone, as a metabolite. Ethene has many physiological effects, among which there are a respiratory promoting effect and maturity promoting effect, and, therefore, largely relates to maturity and also loss of freshness of the plants. The loss of freshness has been a problem especially in the storage or the distribution of vegetables, fruits and flowers. A post harvest preservation to maintain freshness of vegetables, fruits and flowers is therefore highly desirable.

The instant process is particularly useful when the ethene is generated during the storage of fruits, flowers or vegetables.

For example, the iodine doped titaniumdioxid may be used in polymer products, such as plastic films, sheets, bags, bottles, styrofoam cups, plates, utensils, blister packages, boxes, package wrappings, plastic fibers, tapes, twine agricultural films, disposable diapers, disposable garments, shop bags, refuse sacks, cardboard boxes, filtering devices (for refrigerators) and the like. The articles may be manufactured by any process available to those of ordinary skill in the art including, but not limited to, extrusion, extrusion blowing, film casting, film blowing, calendering, injection molding, blow molding, compression molding, thermoforming, spinning, blow extrusion and rotational casting.

In particular, this is of interest in the area of packaging articles, such as films, boxes, filters, labels, bags and sachets. The rate of the gas decomposition can be adjusted by simply changing the concentration of the iodine doped titaniumdioxid and the light exposure time.

Particularly suitable is the incorporation in sachets made from cellulosic materials.

For example the iodine doped titaniumdioxid is incorporated in a natural or synthetic polymer material.

Suitable natural or synthetic polymers are mentioned below.

1. Polymers of monoolefins and diolefins, for example polypropylene, polyisobutylene, polybut-1-ene, poly-4-methylpent-1-ene, polyvinylcyclohexane, polyisoprene or polybutadiene, as well as polymers of cycloolefins, for instance of cyclopentene or norbornene, polyethene (which optionally can be crosslinked), for example high density polyethene (HDPE), high density and high molecular weight polyethene (HDPE-HMW), high density and ultrahigh molecular weight polyethene (HDPE-UHMW), medium density polyethene (MDPE), low density polyethene (LDPE), linear low density polyethene (LLDPE), (VLDPE) and (ULDPE).

Polyolefins, i.e. the polymers of monoolefins exemplified in the preceding paragraph, preferably polyethene and polypropylene, can be prepared by different, and especially by the following, methods:
a) radical polymerisation (normally under high pressure and at elevated temperature).
b) catalytic polymerisation using a catalyst that normally contains one or more than one metal of groups IVb, Vb, VIb or VIII of the Periodic Table. These metals usually have one or more than one ligand, typically oxides, halides, alcoholates, esters, ethers, amines, alkyls, alkenyls and/or aryls that may be either π- or σ-coordinated. These metal complexes may be in the free form or fixed on substrates, typically on activated magnesium chloride, titanium(III) chloride, alumina or silicon oxide. These catalysts may be soluble or insoluble in the polymerisation medium. The catalysts can be used by themselves in the polymerisation or further activators may be used, typically metal alkyls, metal hydrides, metal alkyl halides, metal alkyl oxides or metal alkyloxanes, said metals being elements of groups Ia, IIa and/or IIIa of the Periodic Table. The activators may be modified conveniently with further ester, ether, amine or silyl ether groups. These catalyst systems are usually termed Phillips, Standard Oil Indiana, Ziegler (-Natta), TNZ (DuPont), metallocene or single site catalysts (SSC).

2. Mixtures of the polymers mentioned under 1), for example mixtures of polypropylene with polyisobutylene, polypropylene with polyethene (for example PP/HDPE, PP/LDPE) and mixtures of different types of polyethene (for example LDPE/HDPE).

3. Copolymers of monoolefins and diolefins with each other or with other vinyl monomers, for example ethene/propylene copolymers, linear low density polyethene (LLDPE) and mixtures thereof with low density polyethene (LDPE), propylene/but-1-ene copolymers, propylene/isobutylene copolymers, ethene/but-1-ene copolymers, ethene/hexene copolymers, ethene/methylpentene copolymers, ethene/heptene copolymers, ethene/octene copolymers, ethene/vinylcyclohexane copolymers, ethene/cycloolefin copolymers (e.g. ethene/norbornene like COC), ethene/1-olefins copolymers, where the 1-olefin is generated in-situ; propylene/butadiene copolymers, isobutylene/isoprene copolymers, ethene/vinylcyclohexene copolymers, ethene/alkyl acrylate copolymers, ethene/alkyl methacrylate copolymers, ethene/vinyl acetate copolymers or ethene/acrylic acid copolymers and their salts (ionomers) as well as terpolymers of ethene with propylene and a diene such as hexadiene, dicyclopentadiene or ethylidene-norbornene; and mixtures of such copolymers with one another and with polymers mentioned in 1) above, for example polypropylene/ethene-propylene copolymers, LDPE/ethene-vinyl acetate copolymers (EVA), LDPE/ethene-acrylic acid copolymers (EAA), LLDPE/EVA, LLDPE/EAA and alternating or random polyalkylene/carbon monoxide copolymers and mixtures thereof with other polymers, for example polyamides.

4. Hydrocarbon resins (for example C₅-C₉) including hydrogenated modifications thereof (e.g. tackifiers) and mixtures of polyalkylenes and starch.

Homopolymers and copolymers from 1.)-4.) may have any stereostructure including syndiotactic, isotactic, hemi-isotactic or atactic; where atactic polymers are preferred. Stereoblock polymers are also included.

5. Polystyrene, poly(p-methylstyrene), poly(α-methylstyrene).

6. Aromatic homopolymers and copolymers derived from vinyl aromatic monomers including styrene, α-methylstyrene, all isomers of vinyl toluene, especially p-vinyltoluene, all isomers of ethyl styrene, propyl styrene, vinyl biphenyl, vinyl naphthalene, and vinyl anthracene, and mixtures thereof. Homopolymers and copolymers may have any stereostructure including syndiotactic, isotactic, hemi-isotactic or atactic; where atactic polymers are preferred. Stereoblock polymers are also included.

6a. Copolymers including aforementioned vinyl aromatic monomers and comonomers selected from ethene, propylene, dienes, nitriles, acids, maleic anhydrides, maleimides, vinyl acetate and vinyl chloride or acrylic derivatives and mixtures thereof, for example styrene/butadiene, styrene/acrylonitrile, styrene/ethene (interpolymers), styrene/alkyl methacrylate, styrene/butadiene/alkyl acrylate, styrene/butadiene/alkyl methacrylate, styrene/maleic anhydride, styrene/acrylonitrile/methyl acrylate; mixtures of high impact strength of styrene copolymers and another polymer, for example a polyacrylate, a diene polymer or an ethene/propylene/diene terpolymer; and block copolymers of styrene such as styrene/butadiene/styrene, styrene/isoprene/styrene, styrene/ethene/butylene/styrene or styrene/ethene/propylene/styrene.

6b. Hydrogenated aromatic polymers derived from hydrogenation of polymers mentioned under 6.), especially including polycyclohexylethene (PCHE) prepared by hydrogenating atactic polystyrene, often referred to as polyvinylcyclohexane (PVCH).

6c. Hydrogenated aromatic polymers derived from hydrogenation of polymers mentioned under 6a.).

Homopolymers and copolymers may have any stereostructure including syndiotactic, isotactic, hemi-isotactic or atactic; where atactic polymers are preferred. Stereoblock polymers are also included.

7. Graft copolymers of vinyl aromatic monomers such as styrene or α-methylstyrene, for example styrene on polybutadiene, styrene on polybutadiene-styrene or polybutadiene-acrylonitrile copolymers; styrene and acrylonitrile (or methacrylonitrile) on polybutadiene; styrene, acrylonitrile and methyl methacrylate on polybutadiene; styrene and maleic anhydride on polybutadiene; styrene, acrylonitrile and maleic anhydride or maleimide on polybutadiene; styrene and maleimide on polybutadiene; styrene and alkyl acrylates or methacrylates on polybutadiene; styrene and acrylonitrile on ethene/propylene/diene terpolymers; styrene and acrylonitrile on polyalkyl acrylates or polyalkyl methacrylates, styrene and acrylonitrile on acrylate/butadiene copolymers, as well as mixtures thereof with the copolymers listed under 6), for example the copolymer mixtures known as ABS, MBS, ASA or AES polymers.

8. Halogen-containing polymers such as polychloroprene, chlorinated rubbers, chlorinated and brominated copolymer of isobutylene-isoprene (halobutyl rubber), chlorinated or sulfo-chlorinated polyethene, copolymers of ethene and chlorinated ethene, epichlorohydrin homo- and copolymers, especially polymers of halogen-containing vinyl compounds, for example polyvinyl chloride, polyvinylidene chloride, polyvinyl fluoride, polyvinylidene fluoride, as well as copolymers thereof such as vinyl chloride/vinylidene chloride, vinyl chloride/vinyl acetate or vinylidene chloride/vinyl acetate copolymers.

9. Polymers derived from α,β-unsaturated acids and derivatives thereof such as polyacrylates and polymethacrylates; polymethyl methacrylates, polyacrylamides and polyacrylonitriles, impact-modified with butyl acrylate.

10. Copolymers of the monomers mentioned under 9) with each other or with other unsaturated monomers, for example acrylonitrile/butadiene copolymers, acrylonitrile/alkyl acrylate copolymers, acrylonitrile/alkoxyalkyl acrylate or acrylonitrile/vinyl halide copolymers or acrylonitrile/alkyl methacrylate/butadiene terpolymers.

11. Polymers derived from unsaturated alcohols and amines or the acyl derivatives or acetals thereof, for example polyvinyl alcohol, polyvinyl acetate, polyvinyl stearate, polyvinyl benzoate, polyvinyl maleate, polyvinyl butyral, polyallyl phthalate or polyallyl melamine; as well as their copolymers with olefins mentioned in 1) above.

12. Homopolymers and copolymers of cyclic ethers such as polyalkylene glycols, polyethene oxide, polypropylene oxide or copolymers thereof with bisglycidyl ethers.

13. Polyacetals such as polyoxymethene and those polyoxymethenes which contain ethene oxide as a comonomer; polyacetals modified with thermoplastic polyurethanes, acrylates or MBS.

14. Polyphenylene oxides and sulfides, and mixtures of polyphenylene oxides with styrene polymers or polyamides.

15. Polyurethanes derived from hydroxyl-terminated polyethers, polyesters or polybutadienes on the one hand and aliphatic or aromatic polyisocyanates on the other, as well as precursors thereof.

16. Polyamides and copolyamides derived from diamines and dicarboxylic acids and/or from aminocarboxylic acids or the corresponding lactams, for example polyamide 4, polyamide 6, polyamide 6/6, 6/10, 6/9, 6/12, 4/6, 12/12, polyamide 11, polyamide 12, aromatic polyamides starting from m-xylene diamine and adipic acid; polyamides prepared from hexamethenediamine and isophthalic or/and terephthalic acid and with or without an elastomer as modifier, for example poly-2,4,4,-trimethylhexamethene terephthalamide or poly-m-phenylene isophthalamide; and also block copolymers of the aforementioned polyamides with polyolefins, olefin copolymers, ionomers or chemically bonded or grafted elastomers; or with polyethers, e.g. with polyethene glycol, polypropylene glycol or polytetramethene glycol; as well as polyamides or copolyamides modified with EPDM or ABS; and polyamides condensed during processing (RIM polyamide systems).

17. Polyureas, polyimides, polyamide-imides, polyetherimids, polyesterimids, polyhydantoins and polybenzimidazoles.

18. Polyesters derived from dicarboxylic acids and diols and/or from hydroxycarboxylic acids or the corresponding lactones, for example polyethene terephthalate, polybutylene terephthalate, poly-1,4-dimethylolcyclohexane terephthalate, polyalkylene naphthalate (PAN) and polyhydroxybenzoates, as well as block copolyether esters derived from hydroxyl-terminated polyethers; and also polyesters modified with polycarbonates or MBS.

19. Polycarbonates and polyester carbonates.

20. Polyketones.

21. Polysulfones, polyether sulfones and polyether ketones.

22. Natural polymers such as cellulose, rubber, gelatin and chemically modified homologous derivatives thereof, for example cellulose acetates, cellulose propionates and cellulose butyrates, or the cellulose ethers such as methyl cellulose; as well as rosins and their derivatives.

23. Blends of the aforementioned polymers (polyblends), for example PP/EPDM, Polyamide/EPDM or ABS, PVC/EVA, PVC/ABS, PVC/MBS, PC/ABS, PBTP/ABS, PC/ASA, PC/PBT, PVC/CPE, PVC/acrylates, POM/thermoplastic PUR, PC/thermoplastic PUR, POM/acrylate, POM/MBS, PPO/HIPS, PPO/PA 6.6 and copolymers, PA/HDPE, PA/PP, PA/PPO, PBT/PC/ABS or PBT/PET/PC.

For instance the natural or synthetic polymer material is celluose, a polyolefin, polystyrene or polyester.

Preferred is a process wherein the natural or synthetic polymer material is a packaging material for fruits, flowers or vegetables.

Typically the iodine doped titaniumdioxid is present in an amount of 0.001 to 10% based on the weight of the natural or synthetic polymer material.

The preparation of the iodine doped titaniumdioxide can be carried out using standard operations with commercial starting materials, such as, for example, titanium(IV) n-butoxide (commercial product of Aldrich) and iodic acid (commercial product of Aldrich).

Another aspect of the invention is a composition comprising iodine doped titaniumdioxide incorporated in a natural or synthetic polymer.

Yet further aspects of the invention are the use of a iodine doped titaniumdioxide for the removal of ethene in a gas atmosphere and the iodine doped titaniumdioxide itself.

The iodine doped titaniumdioxide is a powerful photocatalyst, which can also be used for pollutant removal, air cleansing, water purification, treatment of wet waste, odor removal, antimicrobial (e.g. roofing and tiles), anti-septic, anti-dust and anti-fog purposes.

The term wet waste means waste waters, wet solid waste, sludges and polluted air.

The term waste waters, means polluting waste, more or less thick liquids or fluids, such as for example: waste waters deriving from industrial processes and/or productions; sewages deriving from agricultural activities and zootechnical activities, such as drainage waters from breedings, abattoirs, fishing industries; waste waters from civil settlements, such as houses, shops, offices and hospitals; rain waters or washing waters from squares, roads, parking areas, car washes; motorway drainage waters and from refuelling; drainage waters from recycling plants and waste selection, leachates from disposal sites and from garbage cans. By the term solid wet waste, it is understood to mean waste of a different nature such as, for example, domestic and hospital waste, urban solid waste, putrescible organic waste, green waste.

By the term sludges, it is understood to mean solid or semisolid waste deriving from urban, industrial, agricultural zootechnical waste, or decantation sludges from purification processes, for example of a biological type.

By the term polluted air, it is understood to mean air polluted by toxic or malodorous, gaseous or volatile matters, deriving from human activities, from production processes, from biological purification or from processing plants of solid waste. For example, there may be mentioned the ammonia liberated from animal sewages in the breedings, the organic solvents employed in the paints and glues industry and so on.

By the term polluting agents, it is understood to mean each type of toxic or malodorous matter which is harm-ful for the human being and/or the environment, such as, by way of non limiting example: volatile or not volatile organic substances, of a different nature, origin and composition, for example halogenated residues, drugs, oils, greases, surfactants, detergents, fertilizers, solvents; inorganic substances, such as metals, in particular heavy metals, salts; nitrogenous, sulfurous and phosphoric residues. In particular, among the polluting agents, those harmful substances which are not degradable with the known biological purification systems are preferred. One of the aims of the treatment of wet waste is the removal from the same of the polluting agents, in order to eliminate or, at least considerably decrease the possibility of harmful effects on human being and the rest of the ecosystem. General classes of concern include: solvents, volatile organics, chlorinated volatile organics, dioxins, dibenzofurans, pesticides, PCB's, chlorophenols, asbestos, heavy metals, and arsenic compounds. Some specific compounds of interest are 4-chlorophenol, pentachlorophenol, trichloroethylene (TCE), perchloroethylene, CCl₄, HCCl₃, CH₂Cl₂, ethylene dibromide, vinyl chloride, ethylene dichloride, methyl chloroform, p-chlorobenzene, and hexachlorocyclopentadiene. The occurrence of TCE, PCE, CFC-113 (i.e. Freon-113) and other grease-cutting agents in soils and groundwaters is widespread.

The following examples describe the invention.

### EXAMPLES 1 AND 2

Iodine doped titanium dioxides with different iodine amounts.

Iodic acid is dissolved in deionized water and under vigorous stirring tetrabutylorthotitanate is slowly added dropwise at room temperature. The mixture is stirred at room temperature for 24 hours and then the solvents are evaporated. The resulting powder is calcined at 400° C. for 4 h to give the final product as yellow powder. The UV-visible spectra show a shoulder extending in the visible region.

TABLE 1

| | | | | H₂O |
|---|---|---|---|---|
| Example | Code | Ti(OBu)₄ (g/mmol) | HIO₃ (g/mmol) | (ml) |
| 1 | I-TiO₂ (6*) | 51/150 | 4.4/25 | 500 |
| 2 | I-TiO₂ (3*) | 51/150 | 8.8/50 | 500 |

*Ratio Ti(OBu)₄/HIO₃

### APPLICATION EXAMPLES

A given amount of doped TiO₂ (80 mg) is transferred in a Schlenk tube (100 ml) and a certain amount of air/ethene gas mixture is injected in the tube through a rubber cap (approximately 9000 ppm). The composition of the gas mixture contained in the Schlenk tube is monitored over time. To initiate photooxidation, the sample tubes are exposed in a Weatherometer (model ATLAS Ci65A) equipped with a 6500W Xenon lamp (continuous light cycle, black panel temperature=63° C.) for several hours. The results are presented in Table 2.

TABLE 2

| Exposure Time | Ethen in ppm using 80 mg | Ethen in ppm using 80 mg |
|---|---|---|
| [hours] | from Example 1 | from Example 2 |
| 0 | 9178 | 9186 |
| 16 | 4729 | 4435 |
| 40 | 357 | 0 |
| 64 | 0 | 0 |

1. A process for removing ethene from a gas atmosphere, comprising bringing into contact an iodine doped titaniumdioxide
with a gas atmosphere containing at least partly ethene and irradiating the iodine doped titaniumdioxide with light of a wavelength from 300 to 700 nm.

2. A process according to claim 1 wherein the ethene is generated during the storage of fruits, flowers or vegetables.

3. A process according to claim 1 wherein the iodine doped titaniumdioxide is incorporated in a natural or synthetic polymer material.

4. A process according to claim 3 wherein the natural or synthetic polymer material is celluose, a polyolefin, polystyrene or polyester.

5. A process according to claim 3 wherein the natural or synthetic polymer material is a packaging material for fruits, flowers or vegetables.

6. A process according to claim 3 wherein the iodine doped titaniumdioxide is present in an amount of 0.001 to 10% based on the weight of the natural or synthetic polymer material.

7. A composition comprising iodine doped titaniumdioxide incorporated in a natural or synthetic polymer.

8. (canceled)

9. An iodine doped titaniumdioxide.
